(19) United States
(12) Patent Application Publication
(10) Pub. No.: US 2017/0079829 A1
(43) Pub. Date: Mar. 23, 2017

(54) ORTHOTIC FOR PELVIC STABILIZATION

(71) Applicants: Ortho-Team AG, Bern (CH); Universitaet Bern, Bern (CH)

(72) Inventors: Adrian Bosshard, Bolligen (CH); Sandra Widmer, Starrkirch-Wil (CH); Sandro Kohl, Bern (CH); Maurus Johann Baptist Keel, Zuerich (CH); Hendrik Kohlhof, Koeln (DE)

(21) Appl. No.: 15/259,593

(22) Filed: Sep. 8, 2016

(30) Foreign Application Priority Data
Sep. 18, 2015 (CH) 01354/15

(51) Int. Cl. A61F 5/05

(52) U.S. Cl. CPC A61F 5/05

(57) ABSTRACT

An orthotic for pelvic stabilization has a belt unit, which is provided for surrounding the pelvis, and a pull device, which is provided for tightening the belt unit. The belt unit comprises an upper belt strap (1), which surrounds an upper pelvic region, and a lower belt strap (2), which surrounds a lower pelvic region. The pull device comprises a front pull unit with a fastening device, whereby the front pull unit is disposed on a front pelvic region of the belt unit and tightens the upper and the lower belt strap in a front pelvic region by pulling. The fastening device thereby connects together the opposite ends (3, 4, 5, 6) of the upper and of the lower belt strap in the front pelvic region and fastens them under tension. The pull device further comprises a rear pull unit (8, 9), which is disposed in a middle region (7) of the straps on the upper belt strap (1) and on the lower belt strap (2) and is provided for tightening of a rear pelvic region of the belt unit. The rear pull unit is fastened under tension by a fastening unit.

This application claims priority to and the benefit of Swiss Patent Application No. 1354/15 filed on Sep. 16, 2015, the entire disclosure of which as is incorporated by reference herein.

### BACKGROUND

Related Field

The present invention relates to an orthotic for pelvic stabilization having a belt unit around a pelvic region and a pull device for tightening of the belt unit. Such an orthotic is used e.g. for stabilization of pelvic fractures.

Injuries to the pelvis represent about 3% of the accident-related skeletal injuries, and require early recognition during first aid at the accident site since an instability of the pelvis consequently has a great influence on the mortality rate of the accident victim as a result of traumatization. In the case of seriously injured trauma patients, hemorrhagic shock, which is brought about e.g. through rupture of the sacral venous plexus, hemorrhage at the place of fracture or arterial injuries, can quickly lead to death. Hemorrhages caused in this way can be brought under control through a timely stabilization of the pelvis. A sling or a belt is thereby used which is to be placed as early as possible around the pelvis. It has been shown that the tamponade effect thus achieved not only decreases the bleeding, but also reduces the necessary transfusions and stays at the clinic. A pelvic stabilization with a belt can also suffice to achieve sufficient hemodynamic stability for several hours or even days until a definitive treatment can take place.

With a stabilization of the pelvis, the anatomy of the pelvic girdle must be taken into account, which exists with the sacrum positioned in the rear, two aliform ilia, symmetrically adjacent thereto by means of ligaments, and the pubic bones positioned in the front with the pubic symphysis. The robust structure of the pelvis enables the transmission of axial forces from the upper body to the lower extremities, while rotating and vertical forces act upon the pelvis. The pubis region, situated in the front, is susceptible to injuries, which can lead to an opening of the front region of the pelvic girdle. Injuries in the region situated in the rear around the sacrum impair inter alia hemodynamic stability and the nervous system.

Most of the belts used for pelvic stabilization are based on compressing the pelvis from the front side. A belt is thereby brought under the buttocks of the lying patient and pulled tight in order to activate a tensioning mechanism acting upon the front side.

Description of Related Art

Known from EP 1337207 B1 is e.g. an orthotic belt, which can be used with pelvic fractures at the accident site, during transport of the accident victim and also in the emergency room of hospitals. The orthotic belt consists of a wide longitudinal belt element whose ends are connected to each other in the usual way around the patient. In addition, the ends are connected together via a tension device with which the belt can be tightened around the pelvis of the patient. The tension device is formed by a cable pull or pulley system in which cables, via a roller set with a multiplicity of rollers, are led multiple times back and forth between the ends of the belt element and are held at the end by a holder. When pulling at the holder the pulling force distributes itself on the cable running between the belt ends, and pulls the ends evenly toward one another. The width of the belt element thereby covers a wide region of the pelvis, so that the latter is inaccessible for a treatment of the patient. Furthermore the compressive pressure is transferred to the pelvis in an undifferentiated way over the entire width of the belt element, and a fine adjustment for upper and lower pelvic regions is not possible.

Serious injuries often involve also injuries to the rear sacrum region. A compression of a pelvic belt triggered in the front region can thereby lead to an undesired strain on the rear pelvic region. Instability of the rear pelvic region can however easily lead to hemorrhagic shock since the venous plexus is located there.

Known from WO 2014/028248 is a pelvis belt for stabilization in the case of pelvic fractures, which belt comprises two independent, parallel-running belt straps which are tightened separately from one another. An upper strap is supposed to serve for compression in the region of the sacrum and of the front abdomen, and is provided for this purpose at the level of the sacrum. The upper strap has a rear compression pad which presses upon the soft tissue of the sacrum. Furthermore it can have a front compression pad which is supposed to act upon the intersection of the abdominal aorta and the pelvic arteries, in order to reduce the blood flow to more remotely situated arterial regions. A lower strap serves the stabilization of the fracture, and is placed at the level of the head of the femur, for this purpose. The two straps are tightened individually, so that the lower strap can be adjusted for an optimal stabilization of the pelvis and the upper strap for an optimal pressing of the compression pad on the sacrum situated in the rear. Moreover an interim space remains freely accessible between the straps. However the tightening of the straps takes place likewise from the front side, whereby there still exists the above-described risk of an undesired strain on the rear pelvic region. In addition, the fixing of the straps in the tightened position takes place only via a hook-and-loop fastener so that only a very rough adjustment of the pulling force acting on the straps can take place. The compression pads necessary for hemostasis cover over most of the pelvis in the rear region.

### BRIEF SUMMARY

It is an object of the present invention to provide an orthotic for pelvic stabilization which makes possible an optimal stabilization or fixation of different pelvic regions and a reliable hemostasis, which can be put simply and quickly around the pelvis of a patient, which allows compression without great expenditure of energy and which does not impede the further care and treatment of the patient.

This technical problem is solved by the invention through an orthotic for pelvic stabilization according to claim 1. Advantageous embodiments and different embodiment examples are described in the dependent claims.

An orthotic for pelvic stabilization according to the present invention has a belt unit, which is provided for surrounding the pelvis, and a pull device, which is provided for tightening the belt unit. The belt unit comprises an upper and a lower belt strap, which are each wrapped around the pelvis. The upper belt strap surrounds an upper pelvic region and the lower belt strap a lower pelvic region. Upper and lower in this context should be understood with respect to a patient, so that an upper belt strap is closer to the head and preferably comes to be situated around the pelvis in the region of the sacrum, and the lower belt strap is closer to the legs and preferably comes to be situated in the region of the heads of the femur.

The pull device has a front pull unit with a fastening device for fixing of the pull unit under tension. The front pull unit is disposed on a front pelvic region of the belt unit. This means that in a state of surrounding a patient the front pull unit comes to lie on the front pelvic region of the patient, thus e.g. over the pubis region. The front pull unit tightens the upper and the lower belt strap in a front pelvic region by pulling on the pull unit. The front pull unit essentially engages the end regions of the belt straps and reduces, by pulling, the diameter of the belt straps, e.g. at one of the end regions, and thereby the diameter of the belt unit. The front pull unit preferably comprises separate front pull means for the upper and the lower belt strap, so that the upper and the lower belt strap can be tightened separately. The lower belt strap is advantageously wider than the upper belt strap. Preferably the lower strap is one-and-a-half to two-times wider than the upper strap.

Alternatively it is also possible to provide a common front pull means which applies a pull simultaneously on the upper and the lower belt strap.

The fastening device connects together the opposite ends or respectively end regions of the belt strap and the ends or respectively end regions of the lower belt strap in the front pelvic region and fixes them under tension. As an example of a pull unit with fastening device, a passage, e.g. a slit or ring, can be provided on one end region of the belt straps, through which passage the opposite end region is led and tightened. Provided for fastening can be e.g. a closure in the form of a hook-and-loop fastener, which is fixed in a known way by placement of barbed-hook regions and loop regions on top of one another.

When using an orthotic according to the invention, the belt unit, in an open state of the belt straps, is placed as usual around the patient or accident victim in the pelvic region. Each of the oppositely situated end regions of the belt straps are connected with the fastening device so that the upper belt strap and the lower belt strap surround the pelvis. The straps are tightened by means of the front pull unit, and are fixed in the tightened position by means of the fastening device.

According to the invention, the pull device has, in addition to the front pull unit, a rear pull unit and a fastening unit for the rear pull unit. The rear pull unit is disposed on the upper and on the lower belt strap in a middle region of the straps. The rear pull unit is thus situated between the end regions of the straps, upon which the front pull unit acts. If the orthotic is placed around the patient, the rear pull unit comes to be situated on the other side of the patient from the front pull unit and is thereby located on a rear pelvic region. The rear pull unit tightens the belt straps of the belt unit while it is disposed on a rear pelvic region and thereby acts upon the rear pelvic region, whereby the diameter of the belt unit decreases further and compresses the rear pelvic region. The rear pull unit is fixed under tension by means of the fastening unit. A hook-and-loop fastener, for example, can likewise be used for this purpose.

With the orthotic according to the present invention, with placement of the orthotic, a first closing force for stabilization of the pelvis is applied in the front region by means of the front pull unit. By means of the rear pull unit it is possible with the orthotic to exert a constricting closing force also on the rear pelvic region and to thereby achieve a stabilization, which stems bleeding or can even stop hemorrhages in the rear pelvic region. The risk of hemorrhagic shock is thereby significantly reduced compared with conventional orthotics. It is thereby particularly advantageous that the constricting force acts on the upper and the lower belt strap, but at the same time an interim space remains between the straps, so that the rear pelvic region remains accessible for a further examination or treatment without the orthotic having to be taken off. The individual straps of the orthotic act upon specifically selected areas of the pelvis to facilitate an optimal stabilization of the entire pelvic region. Areas which are less relevant for the stabilization can remain free.

In an advantageous embodiment of the orthotic according to the invention, the rear pull unit is disposed in an overlapping way on the upper and lower belt strap, so that the rear pull unit, during tightening of the central belt region, acts at the same time upon the upper and the lower belt strap. The rear pull unit can thereby be tightened with a single hand movement, whereby the patient is ready for transport more quickly.

In an embodiment of the orthotic according to the invention, it is foreseen that the front pull unit has a first front pull means on the upper belt strap for a front pelvic region and a second front pull means on the lower belt strap likewise for a front pelvic region. The first and the second pull means can thereby each comprise an own fastening unit, which in each case connects together and fixes under tension the opposite ends of the upper or respectively lower belt strap in the front pelvic region. Thus, in a first step, the orthotic can be fixed around the patient by means of the front pull unit, and the first and the second front pull means can be held under tension separately from one another. Thus a first force for stabilization of the pelvis can be adjusted separately for the lower and the upper belt strap and adapted to the individual situation.

Foreseen furthermore is that the rear pull unit has an upper pull means, which is situated on the length of the upper belt strap, and a lower pull means, which is situated on the length of the lower belt strap. The upper and the lower pull means are coupled to one another through a force redirection guide, and are able to be actuated by means of a common pull element, such as, for example, a pull cord. Thus the lower and the upper pull means are spaced apart from one another, also during initiation of a pulling force on the rear pull unit. By means of the force redirection, a pulling force which is exerted by pulling on the pull element, will be distributed to the two separate pull means of the rear pull unit. Thus, when putting on the orthotic, the orthotic can be further constricted in a second step through actuation of the pull element and, to be precise, in a rear pelvic region which is of particular importance for the stabilization of the trauma patient. In summary, with the orthotic according to the present invention, a first rough adjustment by means of the front pull unit and a further fine adjustment by means of the rear pull unit take place.

In order to introduce a pulling force into the rear pull unit there preferably exists, with this embodiment, on the length of the lower belt strap, a point of pulling engagement of the common pull element for the upper and the lower pull means. The pelvis is of more stable structure in the region of the lower belt strap than in the region of the upper belt strap, so that a point of pulling engagement in this region brings with it fewer undesired effects on the pelvis with tightening of the orthotic.

Alternatively to this embodiment, it is also possible with the pull device of the orthotic that the first and the second front pull means are coupled, so that the upper and the lower belt strap are able to be pulled tight and fastened jointly. Such an embodiment does accelerate the first step of putting the orthotic around the patient. However the upper and the lower belt strap cannot thereby be tightened independently of one another. Furthermore, alternatively, it is possible for the upper pull means and the lower pull means of the rear pull unit not to be coupled together so that they are able to be acted upon independently of one another with a different pulling force. However this lengthens the time it takes to put on the orthotic.

In an embodiment of the orthotic according to the invention, the rear pull unit is designed as cord pull, or, if the rear pull unit has an upper and lower pull means, upper and lower pull means are designed as cord pull. Such a cord pull unit or such a cord pull means is constructed according to the principle of a pulley. A first cord pull bar is thereby put on one side of the central strap region which is supposed to be pulled together and a second cord pull bar is put on an opposite side of the central strap region on one belt strap, or, in an overlapping way, on both belt straps. The cord pull bars have a multiplicity of deflection points about which a cord or a cable or the like is led multiple times back and forth between the opposite strips and merges into the pull cord for tightening of the cord pull unit or respectively of the cord pull means. Upon tightening of the cord, the necessary force for pulling together of the middle region of the belt strap via the multiple redirection of the cord is distributed to the belt straps so that a minimal pulling force is required for tightening of the belt straps.

According to a preferred variant, the orthotic has an upper cord pull means on the upper belt strap and a lower cord pull means on the lower belt strap, which each have a pull cord for actuation of the cord pull means. The pull cords are preferably coupled to one another and form a joint pull element for actuation of the rear pull unit, with which both cord pull means are able to be tightened at the same time. For this purpose the upper pull cord of the upper cord pull means can e.g. be redirected in the direction of the pulling direction of the lower pull cord of the lower cord pull means, via a force redirection, and can be connected with the lower pull cord. The upper and the lower pull cords thereby form a common pull cord for the rear pull unit. It is thereby ensured that the upper and the lower belt strap are tightened in a way as parallel as possible, even though just one hand movement is necessary for tightening of the rear pull unit.

A cord pull means for an orthotic according to the invention advantageously has a force transmission of at least 1:4 and at most 1:10. With a force transmission of 1:4 the force necessary for constriction of the belt unit is reduced to the extent that, when putting on the orthotic, a rescue worker can produce the desired stabilization of the pelvis without any great physical effort. With a ratio of force transmission of more than 1:10 however a path to be pulled on the pull cord becomes very long, whereby the tightening of the orthotic becomes involved and tedious.

According to a further embodiment of the orthotic according to the invention, a pull cord for actuation of the rear pull unit has an elastic region, which is stretchable in pull direction, and the pull cord has an indicator to indicate the applied pulling force as a function of the stretching of the elastic region. Such a pulling force indicator can be advantageously used with an above-mentioned cord pull as pull unit or also with pull units designed differently, such as a simple pull diversion. With pulling on the pull cord, first the belt unit is tightened until a counterforce is opposed to a further pulling. The further pulling then takes effect on the elastic region of the pull cord, whereby the latter is stretched. The change in length of the elastic region can thus be used as gauge for an applied pulling force. At least part of the elastic region preferably runs under a cover, so that the change in length can be determined relative to the cover. The elastic region is very preferably overlapped by the cover and emerges out of the cover when a predetermined maximal force to be applied has been surpassed, whereby it is indicated that this force has been surpassed. Otherwise it can be ensured by means of a force indicator that a sufficient force is applied for compression and thereby stabilization of the entire pelvis.

In an advantageous embodiment of the orthotic according to the invention, provided in the middle region of the belt straps, i.e. in the region of the rear pull unit, are two support plates for support of the rear pelvic region. The support plates extend beyond the upper and the lower belt strap, and are disposed spaced apart from one another along the belt straps. The support plates are connected together by means of the rear pull unit, and are movable toward one another. The support plates ensure the spacing apart of the upper and lower belt straps, and distribute the compression of the belt unit during tensioning of the straps on the rear pelvic region. Furthermore they support a uniform transfer of the pull of a common pull element on an upper and a lower pull means of the rear pull unit, whereby the support plates move evenly toward one another and constrict and stabilize the rear pelvic region. In other words, the support plates act as push elements which push the left and the right part of the rear pelvic region toward one another, in so doing however ensure that the belt straps remain at the desired spacing with respect to one another and thus in the required position on the pelvis.

The support plates are preferably designed in a mirror-symmetrical way with respect to an axis of the belt unit, whereby the support plates are designed wider in a lower region on the lower belt strap than in an upper region on the upper belt strap. Regions of the pelvis thereby remain free laterally with respect to the support plates, regions which are relevant for examination and treatment of the patient. The region between the support plates also remains free, even though it becomes smaller with tightening of the belt unit, so that the rear pelvic region also remains accessible in the prolongation of the vertebral column.

With a variant of the orthotic, the upper and the lower belt strap are non-continuous in the region between the support plates, and are connected together through an upper cord pull means and a lower cord pull means. This interruption of the belt straps is thus situated in the middle region of the belt straps, which is foreseen for the rear pull unit. For example, the belt straps end at the cord pull bars of the cord pull means. Since the cable or the cord of the cord pull means is significantly narrower compared with the belt strap, a bigger open space thereby remains than with a continuous belt strap.

Through the design of the orthotic with an upper and a lower belt strap as well as support plates between the belt straps in a rear pelvic region there remains at least in the front and lateral pelvic region a freely accessible open space. The pelvis thereby remains accessible for an operative fracture fixation in the front and laterally, without the orthotic having to be removed. Also in the rear pelvic region important areas remain free, compared with pelvic orthotics according to the state of the art, and further care of the patient is not impeded, whereby at the same time an optimal stabilization of the pelvis exists.

The orthotic according to the invention is made of textile materials and has no metal elements whatsoever. The orthotic can therefore remain on the patient during imaging by means of magnetic resonance tomography, and causes no interference with X-ray photographs.

The invention has been presented with reference to a multiplicity of embodiments. The individual technical features of one embodiment can absolutely be used also in combination with another embodiment with the advantages shown. The description of the technical features according to the invention is thus not limited to the respective embodiment.

### BRIEF DESCRIPTION OF THE FIGURES

An advantageous embodiment of the invention will be presented in the following with reference to the drawings, which serve merely explanatory purposes and are not to be interpreted in a limiting way. Features disclosed from the drawings should be considered as belonging to the disclosure of the invention individually and in any combination. In the drawings:

FIG. 1 shows a diagrammatic view of an embodiment of an orthotic for pelvic stabilization according to the invention in an opened state,

FIG. 2 shows a diagrammatic detailed view of a rear pull unit of the embodiment from FIG. 1 in an opened state,

FIG. 3a shows the diagrammatic view of the orthotic from FIG. 1 in the opened state, and

FIG. 3b shows the diagrammatic view of the orthotic from FIGS. 1 and 3a in a tightened state.

### DETAILED DESCRIPTION OF VARIOUS EMBODIMENTS

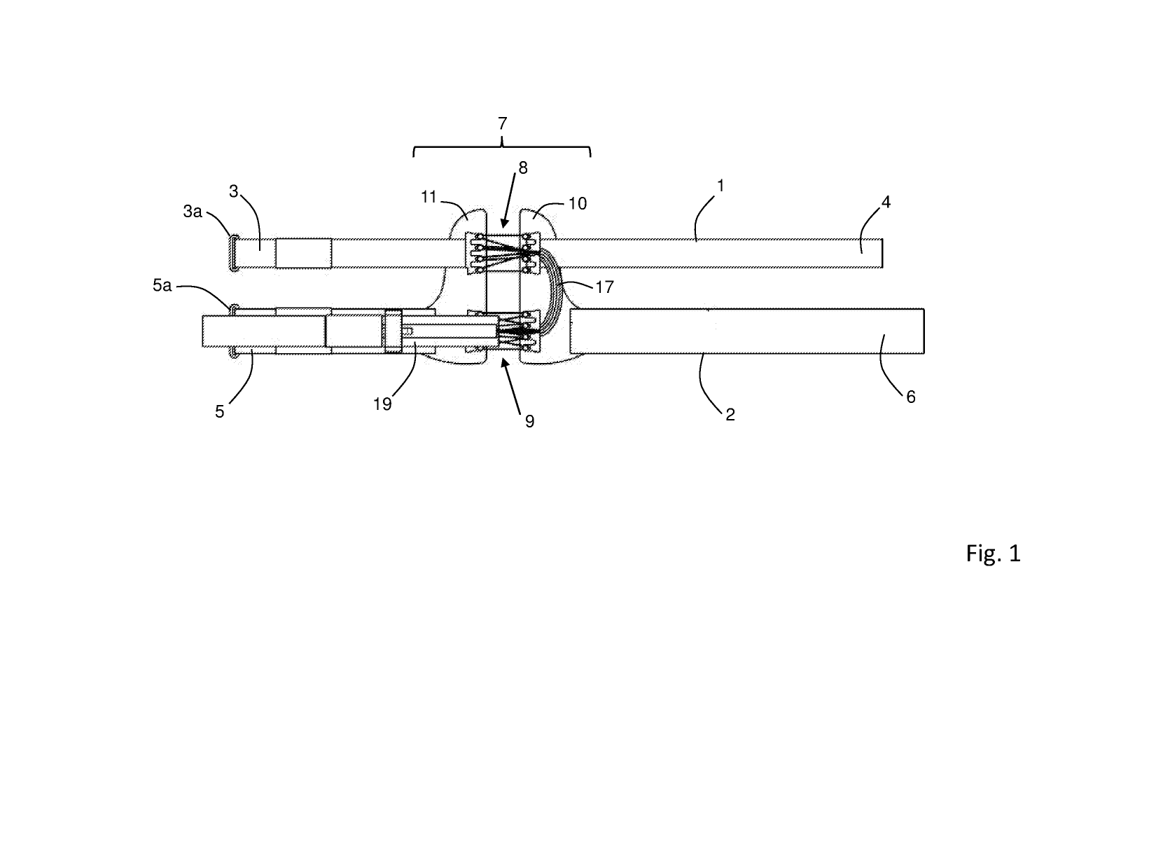

The arrangement of the orthotic on a patient should be used as the frame of reference for describing an embodiment of the orthotic for pelvic stabilization according to the invention with the aid of FIGS. 1, 2, 3a and 3b. As already explained previously, an upper region should be closer to the head and a lower region closer to the legs. Thus the sacrum lies e.g. above the ischium. Furthermore a front region should be situated on a front side of the patient and a rear region on a rear side of the patient. Accordingly e.g. the sacrum is situated in the rear and the pubis in the front.

Shown in FIG. 1 is a diagrammatic representation of an embodiment of an orthotic for pelvic stabilization according to the invention in an opened, stretched state, in order to be able to explain more easily the individual elements of the orthotic. The orthotic comprises a belt unit with an upper belt strap 1 and a lower belt strap 2. The upper belt strap 1 and the lower belt strap 2 run substantially parallel and spaced apart with respect to one another. The lower belt strap 2, which encloses the lower pelvic region, is wider than the upper belt strap 1, which encloses the pelvis in the region of the sacrum. A front pull unit is formed by a first front pull means and a second front pull means. The first front pull means thereby comprises a loop element 3a, such as, for example, a clasp or buckle, which is fixed at one end region 3 of the upper belt strap 1, and the opposite end region 4 of the upper belt strap 1, which can be pulled through the loop element 3a in order to close the upper belt strap 1. Similarly, the second front pull means comprises a further loop element 5a, which is fixed on an end region 5 of the lower belt strap 2, and the opposite end region 6 of the lower belt strap 2, which can be pulled through the further loop element 5a, in order to close the lower belt strap 2. When putting the orthotic on a patient, e.g. the end regions 4 and 6 of the upper and lower belt straps are placed around the pelvis and are pulled through their respective loop element 3a and 5a, so that the belt straps form a loop around the pelvis, whereby the end regions of the belt straps 1, 2 lie on a front pelvic region. The upper belt strap 1 thereby preferably comes to be situated at the level of the sacrum and the lower belt strap 2 at the level of the heads of the femur and the pubis. The end regions 3 and 4 of the upper belt strap 1 thereby form together with the loop element 3a the first front pull means. Similarly the end regions 5 and 6 of the lower belt strap 2 form together with the loop element 5a the second front pull means.

The front pull unit further comprises a fastening device, in order to fasten under tension the upper and the lower belt strap 1, 2 around the pelvis. For this purpose a hook-and-loop fastener, for example, can be provided in that the end regions 4 and 6 at the end of the belt straps have a surface with barbed hooks and adjacent thereto a surface with loops. As soon as an end region 4, 6 with the hook-and-loop fastener is pulled through the respective loop element 3a, 5a, it can be folded over under tension, so that the barbed hook surface can be fixed on the loop surface.

Instead of the loop element and a hook-and-loop fastener, a snap closure or the like could also be used, for example, to form the front pull means, in which a first snap element is attached on the end region 3 or respectively 5 of the belt straps and a second snap element is attached on the opposite end region 4 or respectively 6, the snap elements being able to snap together in a detachable way to form a closure. The snap element on the end region 4 or respectively 6 has an anti-backward-slip safety device, so that this end region is able to be pulled through the snap element in one direction and held firmly, whereby in the opposite direction however a backwards slipping of the end region is blocked.

Provided in a middle region 7 of the belt straps 1 and 2 is a rear pull unit for tightening of the belt unit in the rear pelvic region. In a state of the orthotic being put on the patient, the rear pull unit is situated on the back side of the pelvis. The rear pull unit comprises an upper pull means in the form of an upper cord pull means 8 on the upper belt strap 1 and a lower pull means in the form of a lower cord pull means 9 on the lower belt strap 2. Furthermore two mirror-symmetrically-shaped support plates 10 and 11 are attached in the middle region 7. In a state of the orthotic being put on the patient, the support plate 10 is situated on the right side of the sacrum and the lumbar vertebrae, and the support plate 11 is situated on the left side. With tightening of the middle region 7 by means of the cord pull means 8 and 9, the support plates 10 and 11 are moved toward each other so that the rear pelvic region is compressed and stabilized.

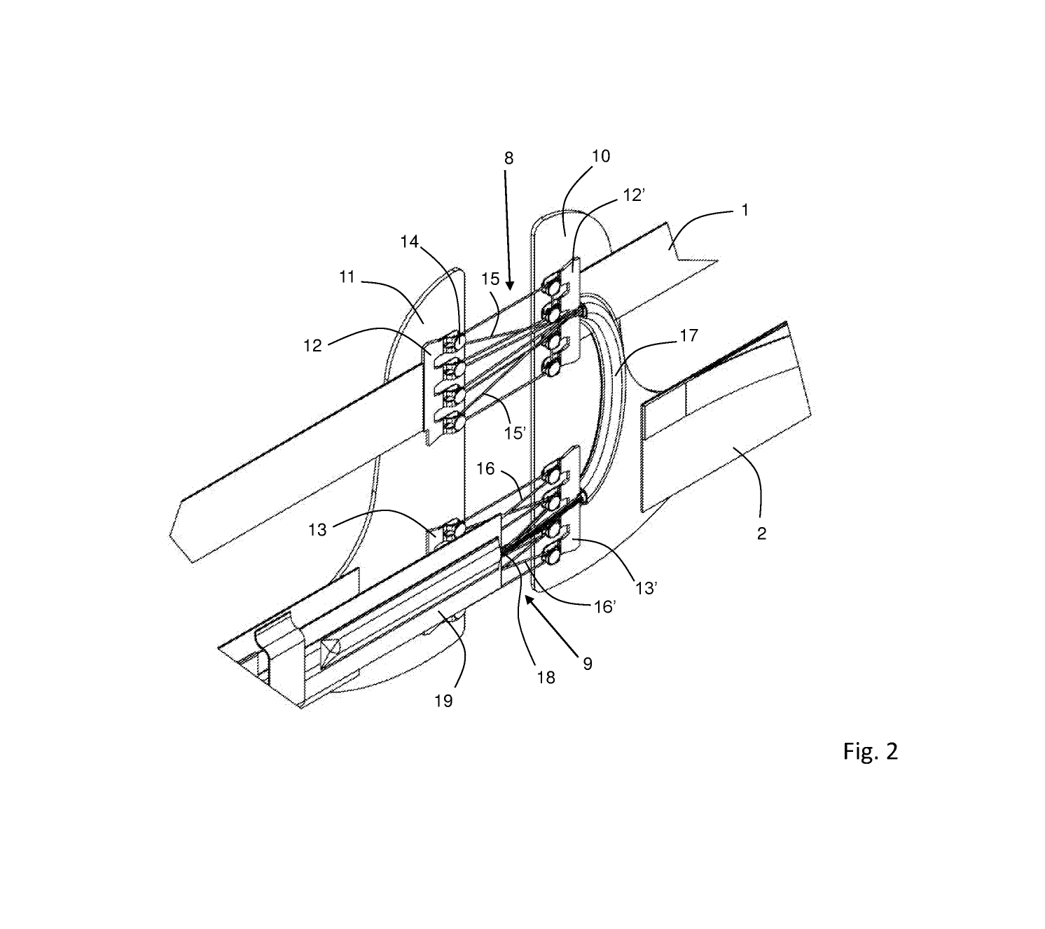

Shown in detail in FIG. 2 is the middle region 7 with the rear pull unit and the support plates 10 and 11. From this figure it can be seen that the belt straps 1 and 2 are interrupted in the region between the support plates 10 and 11, and are connected by means of the cord pull means 8 and 9. The cord pull means 8 has a first cord pull bar 12 on the left support plate 11 and a second cord pull bar 12′ on the right support plate 10; the two strips are fixed to the support plates transversely to the longitudinal direction of the upper belt strap 1 and along its length. Correspondingly the cord pull means 9 has two cord pull bars 13 and 13′, which are both fixed to the support plates transversely to the longitudinal direction of the lower belt strap 2 and along its length. The cord pull bars 12, 12′, 13 and 13′ have a series of several deflection points 14, which are provided for the deflection of one or more pull cables or cords. In the embodiment of the orthotic shown, four deflection points 14 are provided. More or fewer deflection points can also be used, however, as long as a pull cord or cord can be achieved of the pulley type for the rear pull unit.

In the variant shown, two pull cords are used in a cord pull means in order to produce two adjacently situated cord pulls between the cord pull bars. In the case of the upper cord pull means 8, pull cords 15 and 15′ are led between the middle deflection points 14 on the cord pull bar 12′ on the cord pull means 8. The two pull cords 15 and 15′ are led back and forth alternately between the deflection points 14 of the left cord pull bar 12 and the right cord pull bar 12′ until a pulling force ratio 1:4 is present. The pull cord 15 thereby runs around the upper two deflection points 14 of the cord pull bars 12 and 12′ and pull cord 15′ around the lower two deflection points 14 of the cord pull bars 12 and 12′. The pull cords 15 and 15′ are led back between the middle deflection points 14 of the cord pull bar 12′. By means of their guiding between the deflection points 14, the force effect with a tightening of the pull cords 15 and 15′ is distributed over the entire width of the upper belt strap 1. In a comparable way, for the lower cord pull means 9, two pull cords 16 and 16′ are led back and forth alternately between the deflection points 14 of the left cord pull bar 13 and the right cord pull bar 13′, so that, with pulling on the pull cords 16 and 16′, the force effect is distributed over the width of the lower belt strap 2. The deflection points 14 can be designed as simple curvatures on protuberances, or the like, or as rollers. The pull cords bridge the interim space between the support plates at the level of the belt straps and thereby form part of the belt straps.

The pull cords 15 and 15′ enter and exit again the cord pull means 8 at least approximately centrally with respect to the width of the upper belt strap 1. Via a guide 17, the ends of the pull cords 15 and 15′ are diverted from the upper belt strap 1 to the lower belt strap 2 and are guided to the point of pulling engagement 18 of the pull cords 16 and 16′ of the cord pull means 9. The guide can be formed e.g. by a curved channel. The point of pulling engagement 18 of the pull cords 16 and 16′ is situated at least approximately centrally with respect to the width of the belt strap 2. At this point of pulling engagement 18 all pull cords converge and can be tightened in the same direction simultaneously by pulling. From the point of pulling engagement 18 the pull cords form a joint pull element, e.g. in the form of a pull strip 19, with which the upper and the lower cord pull means 8 and 9 are able to be actuated simultaneously. A pulling force acting on the pull strip 19 thereby acts in the same pulling direction on the pull cords 16 and 16′ of the lower cord pull means 9. The pulling force is however redirected, via the guide 17, by 180° to the upper cord pull means 8, so that the pulling direction on the pull cords 15 and 15′ of the upper cord pull means 8 acts in the opposite direction. In summary, the force redirection makes it possible that the lower belt strap 2, through pulling on the lower pull means, is tightened in one pulling direction and the upper belt strap 1, through pulling on the upper pull means, is tightened in the opposite pulling direction. The pulling forces on the lower and upper belt strap thereby act in opposite directions, and the risk is reduced of turning the patient while tightening the rear pull unit, as could be the case with a pull in the same pulling direction.

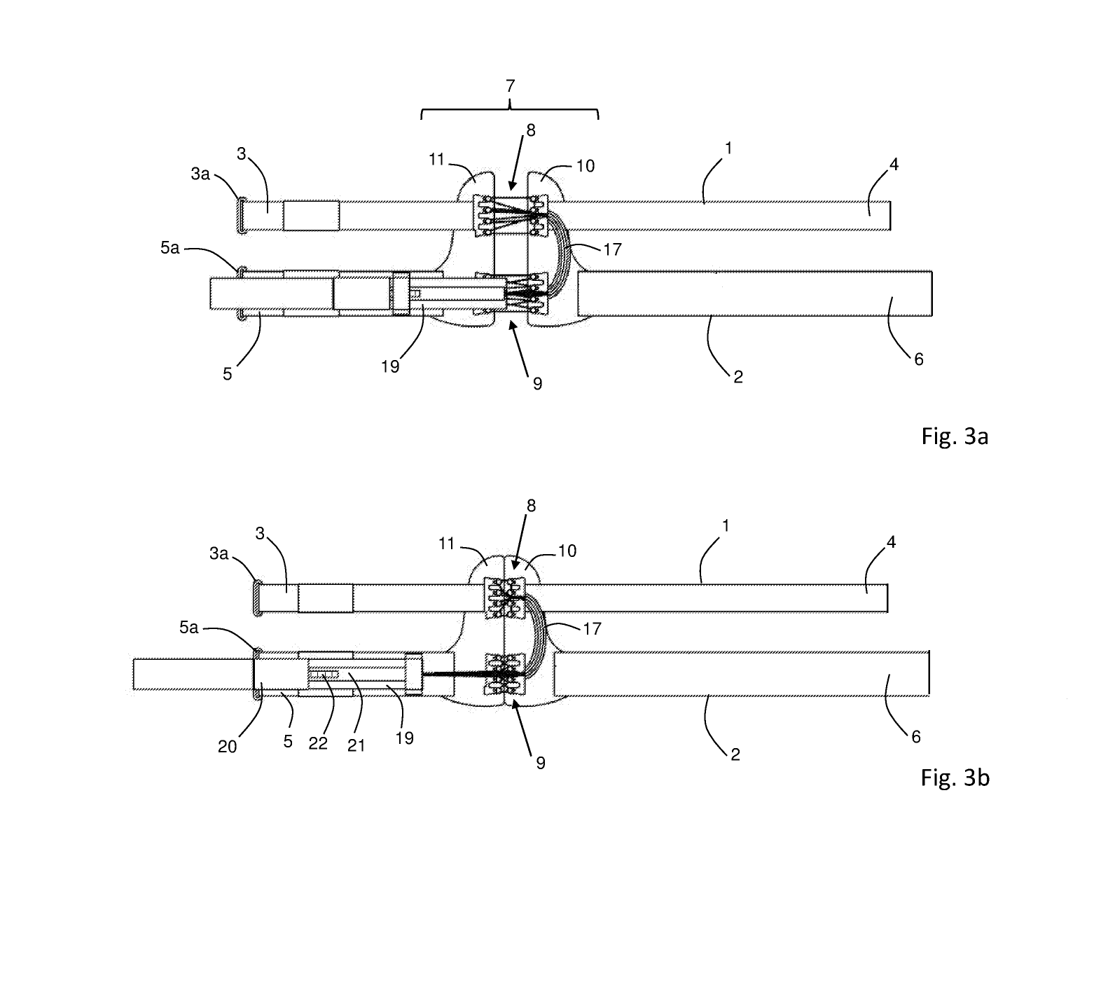

The arrangement of the support plates 10 and 11 can also be seen from FIG. 2. The support plates 10 and 11 extend beyond the upper and the lower belt strap 1 and 2, so that the belt straps 1 and 2 are kept spaced apart from one another. The spacing is at least 5 cm; preferably the spacing is about 10 cm. The support plates 10 and 11 are shown FIGS. 2 and 3a in an opened state of the rear pull unit, in which they are disposed far away from one another. Shown in FIG. 3b is the rear pull unit in a tightened state, in which the support plates 10 and 11 are disposed less far apart from one another; in the shown position the support plates are situated almost adjacent one another, for easier understanding. As described above, the support plates 10 and 11 are connected by means of the rear pull unit, and are movable toward one another, whereby, by means of the joint pull cord, both cord pull means 8 and 9 are able to be tightened evenly and the support plates 10 and 11 move in at least approximately parallel way toward each other. The support plates 10 and 11 support, on the one hand, the belt straps in a way relative to one another, and, on the other hand, they serve the purpose of tightening of the rear pull unit for even distribution of the compressive force on the rear pelvic region.

The support plates 10 and 11 are designed mirror-symmetrical with respect to a longitudinal axis through the belt unit. In a lower region on the lower belt strap 2 the support plates 10 and 11 are designed wider than in an upper region on the upper belt strap 1. Overall the upper belt strap 1 and the lower belt strap 2 and the support plates 10 and 11 are disposed in such a way that in the front and side pelvic region a freely accessible open space is formed between the belt straps and there is as minimal as possible covering in the rear pelvic region by the support plates. The open space between the support plates 10 and 11 is also increased in that the belt straps are interrupted there and are replaced by the pull cords 15, 15′ and 16, 16′. The open space is important for care of the patient, as explained at the beginning.

Shown in FIG. 3a is the rear pull unit of the orthotic in a wide-open state. FIG. 3a corresponds to FIG. 1. The pull strip 19 for actuation of the cord pull means 8 and 9 is not tightened, and is situated in FIG. 3a further to the right. In contrast, the rear pull unit of the orthotic in FIG. 3b is shown in an almost closed state. The pull strip 19 is tightened, and the cord pull means 8 and 9 are tightened, whereby the pull strip 19 in FIG. 3b is situated further to the left. Basically a complete closure of the rear pull unit, so that the support plates 10 and 11 abut one another, is to be avoided, since then a further fine adjustment of the compression force of the orthotic is no longer possible. Although not shown in FIG. 3b, of course in a state in which the orthotic surrounds a patient and has been tightened for stabilization, the upper belt strap 1 and the lower belt strap 2 are closed by means of the loop elements 3a and 5a and the fastening device.

The pull strip 19 has an elastic region 21, which is stretchable in pull direction and in relaxed state lies under a cover 20. The cover 20 is fixed on the pull strip 19. A rubber band can be used in the pull strip as elastic region, for example. The pull strip 19 further comprises an indicator 22 for indication of the applied pulling force as a function of the stretching of the elastic region 21. The indicator 22 can be formed, for example, by the elastic region itself or a marking, e.g. in the form of a color strip, on the elastic region. As can be seen in FIG. 3a, the elastic region 21 lies under the cover 20, when the pull strip 19 is not tightened. Also the indicator 22 is then concealed by the cover 20. If the pull strip 19 is tightened and the pulling force exceeds a predefined force, the elastic region 21 is stretched and emerges out of the cover, so that it becomes visible. It will thereby be indicated to an aid putting the orthotic according to the invention on a patient that the predefined pulling force has been reached and with further pulling will be exceeded. In this way too forceful a tightening of the orthotic can be avoided.

Provided in order to fix the pull strip 19 in a tightened state as in FIG. 3b is a fastening unit, which fixes the pull strip 19 on the lower belt strap 2. A hook-and-loop fastener can again be used as the fastening unit, whereby e.g. a barbed hook surface on the pull strip 19 co-operates with a loop surface on the lower belt strap. Alternatively a hook or button connection could also serve as fastening unit between pull strip 19 and lower belt strap 2.

Reference Numerals

| | |
|---|---|
| 1 | upper belt strap |
| 2 | lower belt strap |
| 3 | end region |
| 3a | loop element |
| 4 | end region |
| 5 | end region |
| 5a | loop element |
| 6 | end region |
| 7 | middle region |
| 8 | upper cord pull means |
| 9 | lower cord pull means |
| 10 | support plate |
| 11 | support plate |
| 12, 12′ | cord pull bar |
| 13, 13′ | cord pull bar |
| 14 | deflection point |
| 15, 15′ | pull cord above |
| 16, 16′ | pull cord below |
| 17 | guide |
| 18 | point of pulling engagement |
| 19 | pull strip |
| 20 | cover |
| 21 | elastic region |
| 22 | indicator |

1. An orthotic for pelvic stabilization, the orthotic comprising:
a belt unit configured for surrounding a user's pelvis and comprising an upper belt strap (1) that surrounds an upper pelvic region, and a lower belt strap (2) that surrounds a lower pelvic region; and
a pull device configured for tightening the belt unit and comprising a front pull unit with a fastening device, a rear pull unit (8, 9), and a fastening unit,
wherein:
the front pull unit is disposed on a front pelvic region of the belt unit and is configured for tightening the upper and the lower belt strap in a front pelvic region by pulling,
the fastening device connects together a opposite ends (3, 4; 5, 6) of the upper and of the lower belt straps in the front pelvic region and fastens them under tension,
the rear pull unit (8, 9) is disposed in a middle region (7) of the straps on the upper belt strap (1) and on the lower belt strap (2) and is configured for tightening of a rear pelvic region of the belt unit, and
the fastening unit is configured for fastening the rear pull unit under tension.

2. The orthotic according to claim 1, wherein the rear pull unit (8, 9) is disposed in an overlapping way on the upper belt strap (1) and lower belt strap (2), so that the rear pull unit during tightening of the central belt region (7) at the same time acts upon the upper and the lower belt strap.

3. The orthotic according to claim 1, wherein the front pull unit has a first front pull means (3a, 4) on the upper belt strap (1) for a front pelvic region and a second front pull means (5a, 6) on the lower belt strap (2) likewise for a front pelvic region.

4. The orthotic according to claim 1, wherein the rear pull unit has an upper pull means, which is situated on the length of the upper belt strap (1), and a lower pull means, which is situated on the length of the lower belt strap (2), whereby the upper and the lower pull means are coupled to one another through a force redirection means (17) and are able to be actuated by means of a common pull element (19).

5. The orthotic according to claim 4, wherein a point of pulling engagement (18) of the common pull element (19), in order to conduct a pulling force into the rear pull unit (8, 9), is provided on the length of the lower belt strap (2).

6. The orthotic according to claim 1, wherein at least one of the rear pull unit or the upper and lower pull means is designed as a cord pull.

7. The orthotic according to claim 6, wherein an upper cord pull means (8) and a lower cord pull means (9) of the rear pull unit are coupled together via at least one common pull element (19).

8. The orthotic according to claim 6, wherein an upper pull cord (15, 15′) for the upper cord pull means (8) is led via a force redirection guide (17) to a lower pull cord (16, 16′) for the lower cord pull means (9), and the upper and the lower pull cords merge into the common pull element (19).

9. The orthotic according to claim 6, wherein a cord pull means (8, 9) has a force transmission of at least 1:4 and at most 1:10.

10. The orthotic according to claim 1, wherein a pull element (19) for actuation of the rear pull unit (8, 9) has an elastic region (21), which is stretchable in pull direction, and the pull element (19) has an indicator (22) to indicate the applied pulling force as a function of the stretching of the elastic region (21).

11. The orthotic according to claim 10, wherein at least one area of the elastic region (21) of the pull element (19) runs under a cover (20).

12. The orthotic according to claim 1, wherein provided in the middle region (7) of the belt straps are two support plates (10, 11) for support of the rear pelvic region, which extend beyond the upper belt strap (1) and the lower belt strap (2), are disposed spaced apart from one another, and are connected by means of the rear pull unit (8, 9) and are movable toward one another.

13. The orthotic according to claim 12, wherein the support plates (10, 11) are designed in a mirror-symmetrical way with respect to an axis of the belt unit, whereby the support plates (10, 11) are designed wider in a lower region on the lower belt strap (2) than in an upper region on the upper belt strap (1).

14. The orthotic according to claim 12, wherein the upper belt strap (1) and the lower belt strap (2) are non-continuous in the region between the support plates (10, 11), and each belt strap (1, 2) is connected together through an upper cord pull means (15, 15′) or respectively a lower cord pull means (16, 16′).

15. The orthotic according to claim 1, wherein the upper belt strap (1) and the lower belt strap (2), and, where applicable, the support plates (10, 11), are disposed in such a way that a freely accessible open space is formed at least in the front and lateral pelvic region.
